(19) United States
(12) Patent Application Publication
(10) Pub. No.: US 2026/0334898 A1
(43) Pub. Date: Oct. 29, 2026

(54) MOBILE RADIOGRAPHIC IMAGING APPARATUS, RADIOGRAPHIC IMAGING METHOD, AND RECORDING MEDIUM

(71) Applicant: Konica Minolta, Inc., Tokyo (JP)

(72) Inventors: Nobuyuki MIYAKE, Kanagawa (JP); Naoki HAYASHI, Tokyo (JP)

(21) Appl. No.: 19/653,398

(22) Filed: Apr. 21, 2026

(30) Foreign Application Priority Data
Apr. 23, 2025 (JP) 2025-071509

(51) Int. Cl. A61B 6/00; A61B 6/10; A61B 6/46

(52) U.S. Cl. CPC A61B 6/4405; A61B 6/107; A61B 6/46; A61B 6/547

(57) ABSTRACT

The mobile radiographic imaging apparatus is a mobile radiographic imaging apparatus that performs radiographic imaging on a subject, and includes a hardware processor that generates a notification for presenting an action taken by an operator during the radiographic imaging based on information on an operation process of the mobile radiographic imaging apparatus, and an output section that outputs the notification.

### CROSS REFERENCE TO RELATED APPLICATIONS

The entire disclosure of Japanese Patent Application No. 2025-071509 filed on Apr. 23, 2025 is incorporated herein by reference in its entirety.

### BACKGROUND

### Technical Field

The present invention relates to a mobile radiographic imaging apparatus, a radiographic imaging method, and a recording medium .

### Description of Related Art

Conventionally, as a radiographic imaging apparatus that performs imaging using radiation such as X-rays, a mobile radiographic imaging apparatus called a medical cart has been put into practical use. Such a mobile radiographic imaging apparatus can be used even in a space (for example, an Intensive Care Unit (ICU), a general hospital room, or the like) in which a mechanism for radiation shielding or protection is not provided, outside a radiation management area such as a radiographic imaging room.

In a case where imaging is performed in the space such as an ICU or a general hospital room in which a mechanism for radiation shielding or protection is not provided, many work processes are required before radiographic imaging. Examples of the necessary work processes include setting imaging conditions in advance in consideration of the state of the hospital room, preparing in advance a protective material or the like necessary for radiographic imaging, and considering the placement of a medical cart.

In addition, when imaging is performed in the ICU or the general hospital room, there is a possibility that a person such as a medical worker such as a nurse or a family of a patient who is a subject exists nearby. Therefore, it is necessary to perform preparation and measures in consideration of radiation exposure to persons present nearby.

For example, Japanese Unexamined Patent Publication No. 2014-236798 discloses an X-ray diagnostic apparatus that displays a dose map of a subject model and a scattered radiation map in the vicinity of the subject model, which are formed according to X-ray generation conditions, to enable easy management of radiation exposure to a practitioner. By causing the dose map to be displayed in this way, it is possible to cause the practitioner who is an operator to grasp the distribution of the dose in real time.

### SUMMARY

However, in the technique described in Japanese Unexamined Patent Publication No. 2014-236798, the dose map is displayed, and thus it is possible to cause the practitioner who is the operator to grasp the surrounding radiation dose. However, at this time, there is a problem in that it is not possible to appropriately grasp what action the operator should specifically take, and thus it is not possible to efficiently perform imaging. In addition, when the operator is unfamiliar with imaging, the dose map cannot be appropriately used.

Objectives of the present invention include providing a mobile radiographic imaging apparatus, a radiographic imaging method, and a recording medium which enable an operator to appropriately work at the time of radiographic imaging.

In order to achieve at least one of the above-described objectives, a mobile radiographic imaging apparatus reflecting one aspect of the present invention is a mobile radiographic imaging apparatus that performs radiographic imaging on a subject, including:
a hardware processor that generates a notification for presenting an action taken by an operator during the radiographic imaging based on information regarding an operation process of the mobile radiographic imaging apparatus; and
an output section that outputs the notification.

A radiographic imaging method reflecting another aspect of the present invention is a radiographic imaging method for a mobile radiographic imaging apparatus that performs radiographic imaging on a subject,the method including:
generating a notification for presenting an action taken by an operator during the radiographic imaging based on information regarding an operation process of the mobile radiographic imaging apparatus;and
outputting the notification.

A non-transitory computer-readable recording medium reflecting still another aspect of the present invention stores therein a program for causing a computer to execute the above-described radiographic imaging method.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of a medical cart in use;

FIG. 2 is a diagram illustrating an example of the medical cart while moving or not in use;

FIG. 3 is a diagram illustrating functional blocks of the medical cart; and

FIG. 4 is a flowchart for explaining an example of a flow of operation of the medical cart according to the present embodiment.

### DETAILED DESCRIPTION OF EMBODIMENTS

Hereinafter, one or more embodiments of the present invention will be described with reference to the drawings. However, the scope of the invention is not limited to the disclosed embodiments.

### 1. Radiographic Imaging System

First, a schematic configuration of a radiographic imaging apparatus according to an embodiment of the present disclosure will be described. The radiographic imaging apparatus includes at least a radiation generating apparatus. The radiographic imaging apparatus may be, for example, the medical cart 100. The medical cart 100 may have an automatic driving function. The radiographic imaging apparatus may be a stationary radiographic imaging apparatus.

Further, the radiographic imaging apparatus may be, for example, a radiographic imaging apparatus in which a radiation generating apparatus having a tube is placed at one tip of an arm formed in a C shape so as to surround a patient to be imaged, and a radiation detection apparatus is placed at the other tip. The radiographic imaging apparatus may be fixed to the arm or may be detachable.

FIG. 1 is a diagram illustrating an example of a medical cart 100 in use.

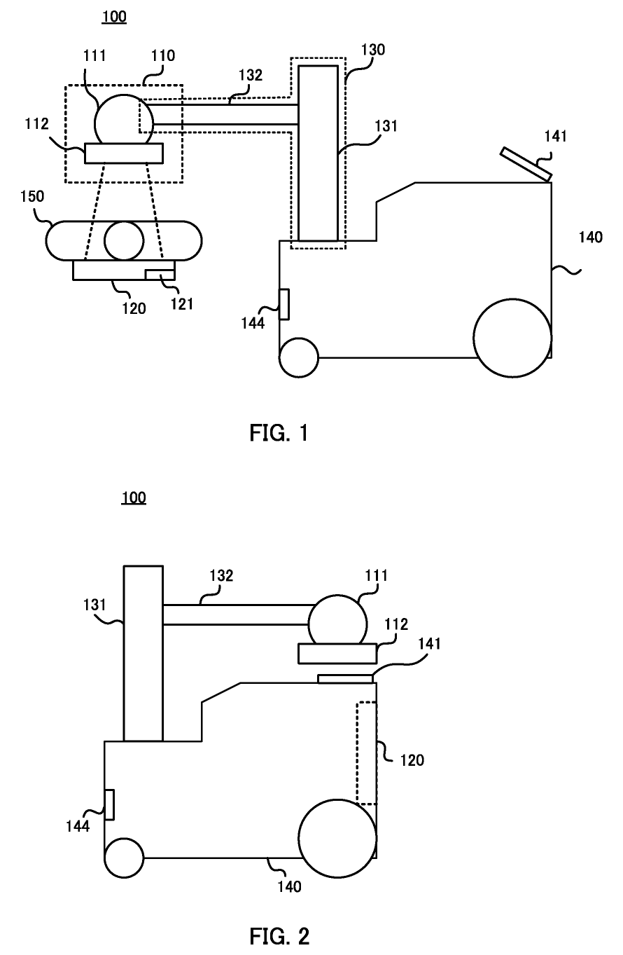

FIG. 2 is a diagram illustrating an example of the medical cart 100 when the medical cart 100 is moved or not used.

The medical cart 100 includes a radiation generating apparatus 110, a radiation detection apparatus 120, an arm 130, and a main body section 140.

The medical cart 100 may be communicable with a Hospital Information System (HIS), a Radiology Information System (RIS), an image analysis device, a Picture Archiving and Communication System (PACS), another radiographic imaging apparatus, and the like, which are not illustrated.

The medical cart 100 may be capable of setting a plurality of imaging modes including a dynamic imaging mode for dynamic imaging and a fluoroscopic imaging mode for fluoroscopic imaging.

### Radiation Generating Apparatus

The radiation generating apparatus 110 includes a generator 111 and a collimator 112. The radiation generating apparatus 110 may be a radiation generating unit.

The generator 111 includes a tube. When a voltage is applied to the tube and a current flows through the tube, radiation, for example, X-rays are emitted from the tube. A pulsed voltage may be applied to the tube, and pulsed radiation, for example, X-rays may be emitted from the tube.

The maximum amount of radiation that the generator 111 emits, that is, the allowed amount of radiation is limited in accordance with an imaging mode (e.g., a dynamic imaging mode or a fluoroscopic mode) of moving image capturing set in the medical cart 100. In the dynamic imaging mode, the amount of radiation to be emitted is limited due to the limitation imposed for use as a general imaging apparatus. In the fluoroscopic mode, since the generator is not subject to limitations imposed for use as a general imaging apparatus, a larger amount of radiation can be emitted than in the dynamic imaging mode. In the fluoroscopic mode, the radiation dose may not be limited. The generator 111 emits radiation based on the setting from the input section. The imaging conditions are, for example, conditions related to the subject 150 such as an imaged site, an imaging direction, and a physique, and conditions related to radiation emission such as an imaging time, a tube voltage, a tube current, a radiation time (irradiation time) per frame, a tube current-time product (mAs value), a frame rate, the allowable number of frames (the maximum number of frames that can be imaged per imaging), a pulse width, a pulse interval, a pulse period, a pulse duty ratio, Focus to Object Distance (FOD), Source to Image-receptor Distance (SID), and an irradiation field range (the aperture of the collimator 112).

In dynamic imaging, that is, imaging in the dynamic imaging mode, the radiation generating apparatus 110 repeatedly emits radiation pulses in a cycle of a plurality of times per unit time (e.g., 15 times per second) for a predetermined time (duration) while an emission instruction is given. Here, the pulse period and the duration are set in advance by the input/output section 141. The pulse interval, the pulse width, and the duty ratio of the pulse may be set in advance by the input/output section 141. The radiation detection apparatus 120 acquires an image corresponding to each pulse to acquire a series of multiple images (dynamic images). Although a configuration in which radiation pulses are emitted in the dynamic imaging mode according to the present embodiment will be described as an example, a configuration in which radiation is continuously emitted may be employed. In the dynamic imaging mode, emission of radiation is limited due to limitations imposed for use as a general imaging apparatus.

Fluoroscopy, that is, imaging in the fluoroscopic mode, is repeated by the radiation generating apparatus 110 at a cycle of a plurality of times per unit time (for example, 6 times, 10 times, or 15 times per second) while an instruction to emit a radiation pulse is given. Here, the pulse period is set in advance by the input/output section 141. In the fluoroscopic mode, no duration is set. That is, in the fluoroscopic mode, the emission of the radiation is not limited by the duration. Further, in the fluoroscopic mode, since there is no limitation imposed for use as a general imaging apparatus, more radiation can be emitted than in the dynamic imaging mode. In the fluoroscopic mode, the radiation dose may not be limited. The pulse interval, the pulse width, and the duty ratio of the pulse may be set in advance by the input/output section 141. The radiation detection apparatus 120 detects an image corresponding to each pulse, and the input/output section 141 displays the image. Note that a configuration in which a radiation pulse is emitted in the fluoroscopic mode in the present embodiment will be described as an example, but a configuration in which radiation is continuously emitted may be employed.

The collimator 112 narrows an irradiation field of the emitted radiation. The collimator 112 may include a shielding means (exposure reducing means) such as an additional filter. The shielding means may narrow a region to be irradiated with the emitted radiation. The shielding means may weaken the intensity of the emitted radiation. The shielding means may weaken the intensity of a low energy band of the emitted radiation.

### Radiation Detection Apparatus

The radiation detection apparatus 120 includes a communication section 121. In addition, although not illustrated, a sensor board, a scanning circuit, a readout circuit, a controller, and the like are provided. The radiation detection apparatus 120 may be a radiation detection unit.

The radiation detection apparatus 120 detects radiation emitted from the radiation generating apparatus 110 through the subject 150. The subject 150 is, for example, a human, an animal, or the like.

On the sensor board, the pixels are arranged two dimensionally (in a matrix). Each pixel includes a radiation detection element that generates an electric charge corresponding to the dose of radiation received through a subject and a switch element that accumulates and discharges the electric charge. The scanning circuit turns on or off each switch element. The readout circuit reads out the amount of charge emitted from each pixel as a signal value (intensity). The controller controls the entire radiation detection apparatus 120. The controller generates a radiographic image from the plurality of signal values read by the readout circuit. The communication section 121 transmits the data of the radiographic image generated by the readout circuit, various signals, and the like to the outside. Further, the communication section 121 receives various kinds of information and various signals. The communication section 121 may perform wireless communication or may perform wired communication.

In each pixel of the radiation detection apparatus 120 configured in such a manner, when the radiation detection element receives radiation in a state in which the scanning circuit turns off the switch element, the radiation detection element generates electric charge corresponding to the dose of radiation and accumulates the electric charge. When the scanning circuit turns on the switch element, the accumulated charge is released, and the readout circuit detects the amount of charge released from each pixel and generates a signal value indicating the amount of charge. The controller generates a radiographic image based on the signal value generated for each pixel. The generated individual radiographic images are one still image in the dynamic imaging. In the case of pulsed radiation, one still image is generated for each pulse.

The communication section 121 communicates with the communication section 144 of the main body section 140 and transmits the generated radiographic image to the communication section 144 of the main body section 140. The communication section 121 may transmit the still image to the main body section 140 each time one still image is generated, or may collectively transmit a plurality of still images to the communication section 144 of the main body section 140. The communication section 121 may communicate with other than the main body section 140. The communication performed by the communication section 121 may be wireless communication or wired communication.

When the radiation generating apparatus 110 performs pulse emission, the timing at which the radiation detection apparatus 120 generates a plurality of still images constituting a dynamic image is synchronized with the timing at which radiation is emitted from the radiation generating apparatus 110. On the other hand, in a case where the radiation generating apparatus 110 performs continuous emission, the timing at which the radiation detection apparatus 120 generates the plurality of still images forming the dynamic image is any timing during the time of the continuous emission.

The radiation detection apparatus 120 may be stored in a storage section provided in the main body section 140 when the medical cart 100 is moved or not in use. A state in which the radiation detection apparatus 120 is stored in the storage section of the main body section 140 is illustrated by a dotted line in FIG. 2. When the radiation detection apparatus 120 is stored in the storage section of the main body section 140, the radiation detection apparatus 120 may be connected to the main body section 140 in a wired manner, and charging of the radiation detection apparatus 120 from the main body section 140 and communication with the main body section 140 may be performed. When the radiation detection apparatus 120 is stored in the storage section of the main body section 140, software or firmware of the radiation detection apparatus 120 may be updated by communication with the main body section 140. Charging of the radiation detection apparatus 120 and communication with the main body section 140 may be performed wirelessly.

The radiation detection apparatus 120 may be wirelessly connected during use. The radiation detection apparatus 120 may be driven by an internal battery. Power may be supplied to the radiation detection apparatus 120 from the main body section 140 in a wired or wireless manner.

The radiation detection apparatus 120 may be connected to the main body section 140 in a wired manner when being moved or not being used, and may be connected to the main body section 140 in a wireless manner when being used.

### Arm

The arm 130 includes a vertical arm 131 and a horizontal arm 132. The vertical arm 131 supports the horizontal arm 132 so as to be pivotable, that is, rotatable with respect to the Z axis. Furthermore, the vertical arm 131 may movably support the horizontal arm 132. The horizontal arm 132 supports the radiation generating apparatus 110 so as to be rotatable, that is, rotatable with respect to the X-axis and the Y-axis. The radiation generating apparatus 110 can be oriented in any direction with respect to the main body section 140 by the vertical arm 131 and the horizontal arm 132. The position and orientation of the arm may be determined by input from the input/output section 141. The vertical arm 131 and the horizontal arm 132 may be rotatable and movable by control from the main body section 140 or may be rotatable and movable manually.

Furthermore, the arm 130 is not limited to this example, but may be formed in, for example, a C shape. In this case, the radiation generating apparatus 110 having a tube is placed at one end of the arm 130, and the radiation detection apparatus 120 is placed at the other end. The radiation detection apparatus 120 disposed on the arm 130 may be fixed to the arm 130 or may be detachable.

### Main Body Section

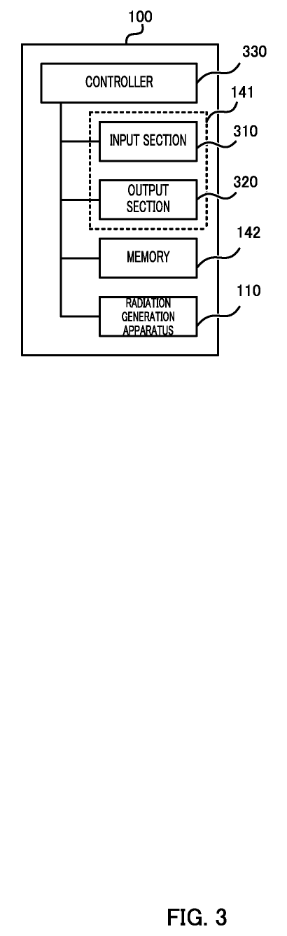

The main body section 140 includes the input/output section 141 and the communication section 144. The main body section 140 may include a controller (control circuit) 330 and a memory 142 illustrated in FIG. 3. Although not illustrated, the main body section 140 may include the storage section that stores the radiation detection apparatus 120.

The input/output section 141 can change an inclination angle with respect to the main body section 140. The input/output section 141 may be separable from the main body section 140. When the input/output section 141 is separated from the main body section 140, the input/output section 141 and the main body section 140 are connected to each other in a wireless or wired manner.

The input/output section 141 may include a touch screen, a keyboard, a mouse, a microphone, a camera, a display, a speaker, a display lamp, and the like. The input/output section 141 may be divided into an input section and an output section. As the input/output section 141, input means such as an input by voice, an input by gesture, an input by line of sight, and an input by brain waves may be available. The input/output section 141 performs setting of the radiation generating apparatus 110. The input/output section 141 can perform an operation of instructing the radiation generating apparatus 110 to perform radiation. The input/output section 141 may output the radiation instruction during a period from when a toggle button is pressed for the first time to when the toggle button is pressed for the second time, or may output the radiation instruction during a period in which the button is continuously pressed.

Imaging conditions are input to the input/output section 141 for dynamic imaging. The imaging conditions include an imaging time, a frame rate, a tube voltage, a tube current, a radiation time (irradiation time) per frame, or a tube current-time product (mAs). The imaging conditions may be set based on an examination order received from the RIS or the like. The imaging conditions are set in the radiation generating apparatus 110.

The examination order information includes various kinds of information about dynamic imaging to be executed next, such as patient information, examination information, and data attributes. The patient information is information related to a subject to be imaged, and is information including at least one of, for example, the height, weight, age, sex, and Body Mass Index (BMI) of the subject. The examination information includes information such as an examination ID, an examination target site, and a type of analysis. Further, the examination order information may include information relating to an imaging location such as a location at which radiographic imaging is performed (a type such as an ICU or a general hospital room, a specific room name, or the like) and an attribute of the location at which radiographic imaging is performed (a layout of the room, placement of a patient bed, or the like).

An external factor is input to the input/output section 141 during dynamic imaging. The external factor may be generated by, for example, analyzing an image captured by a camera mounted on the medical cart 100 or information acquired by a sensor mounted on the medical cart 100. The external factor generated based on the captured image or the information acquired by the sensor may be modifiable.

In response to an operation of the input/output section 141, the generator 111 emits radiation of a dose set by the input/output section 141. The radiation is, for example, X-rays.

In addition, the input/output section 141 displays various notifications, and an operator performs an operation in response to the notifications. Details of the notification will be described later.

Note that the input/output section 141 may be placed in the radiation generating apparatus 110 in addition to the main body section 140. This is to enable an operator, such as a technician, to perform operation and check various kinds of display when the operator moves the medical cart 100. In the following description, when the input/output section 141 is specified as an input/output section placed in the radiation generating apparatus 110, the input/output section 141 may be referred to as a "tube side input/output section".

The communication section 144 communicates with the radiation detection apparatus 120. Communication between the communication section 144 of the main body section 140 and the communication section 121 of the radiation detection apparatus 120 may be wireless communication or wired communication. The communication section 144 performs communication with the RIS or the like. For example, the communication section 144 receives an examination order from the RIS by wireless communication.

### Functional Blocks

FIG. 3 is a diagram illustrating functional blocks of the medical cart 100. The medical cart 100 includes an input section 310, an output section 320, a memory 142, a radiation generating apparatus 110, and a controller 330. The input section 310 and the output section 320 form the input/output section 141. The input section 310 and the output section 320 may be integrated.

When the imaging start is instructed, the input section 310 may notify the controller 330 that the imaging start has been instructed.

The output section 320 displays various notifications to an operator such as a technician by the controller 330. In addition, a notification regarding the scattered radiation dose may be displayed on the output section 320.

The memory 142 stores a program, a parameter, and the like used by the controller 330 for processing. The memory 142 may store a map (hereinafter, referred to as a base map) in which a layout of an imaging location, placement of a patient bed, and the like are stored. The memory 142 may store a two dimensional base map and a three dimensional base map. The memory 142 may store a base map corresponding to a case where imaging is performed in a sitting position and a base map corresponding to a case where imaging is performed in a lying position. The memory 142 may store a base map in which nothing is set.

The controller 330 controls the entire medical cart 100. The controller 330 is constituted by a computer such as a Central Processing Unit (CPU), an Application Specific Integrated Circuit (ASIC), or a Field Programmable Gate Array (FPGA). The CPU is an example of a hardware processor that functions as the controller 330.

In the present embodiment, the controller 330 presents, via the input/output section 141 (output section 320), an action to be taken by an operator such as a technician at the time of radiographic imaging, based on information on the operation process of the medical cart 100. Specifically, the controller 330 generates various notifications for presenting the operator's action, using the input/output section 141 (the output section 320).

For example, the controller 330 notifies the operator of preparation or wearing of protective clothing, placement of the medical cart 100, placement of a partition (protective material) for an imaging location, and the like. Specific notification by the controller 330 will be described later.

The controller 330 may be instructed to start imaging from the input section 310. The controller 330 may exist in the main body section 140.

The controller 330 may select and acquire a base map corresponding to the location where the medical cart 100 is present from the memory 142. The controller 330 may select and acquire a two dimensional base map or a three dimensional base map in accordance with an input from the input section 310.

The controller 330 estimates the scattered radiation dose based on the imaging conditions, and causes the output section 320 to display the estimation result in advance before imaging. The controller 330 calculates the amount of radiation to be emitted, based on the imaging time, the tube voltage, the tube current, the radiation time (irradiation time) per frame, the tube current-time product (mAs value), the frame rate, the allowable number of frames (the maximum number of frames that can be imaged per imaging), the pulse width, the pulse interval, the pulse period, the duty ratio of the pulse, and the like. The controller 330 calculates a region to be irradiated based on the imaged site, the imaging direction, the FOD, the SID, the patient body thickness, the irradiation field range, and the like.

As the FOD and the SID, for example, values derived from a value of a distance measuring sensor attached to the collimator 112 may be used. Further, when the distance measuring sensor is not attached to the collimator 112 or when an abnormality occurs in the distance measuring sensor, for example, values included in the imaging order may be used as the FOD and the SID. For the irradiation field range, for example, the state of the aperture of the collimator 112 acquired from the collimator 112 may be used.

Furthermore, the controller 330 can estimate the scattered radiation dose that is scattered to a region other than the region to be irradiated, based on the amount of radiation to be emitted and the region to be irradiated. The controller 330 can estimate the amount of reflection and absorption of the emitted radiation based on the layout of the imaging location, conditions related to the subject 150 such as physique, the placement of the patient bed, the position of the shielding plate, the material of the shielding plate, and the like. The controller 330 may estimate the scattered radiation dose that is scattered to a region other than the irradiated region, in consideration of the amount of reflected and absorbed radiation in addition to the amount of emitted radiation and the irradiated region.

The controller 330 may display the estimation result in response to the imaging start instruction input from the input section 310, and notify the operator and surrounding persons to take shelter.

Here, since there are various external factors such as the layout of the imaging location, the placement of the patient bed, and the shielding plate in imaging by the medical cart outside the radiation management area, it is necessary to present an appropriate evacuation place to the operator or surrounding persons. Therefore, the controller 330 may be allowed to provide an instruction to start imaging when it is confirmed that the practitioner and nearby people have moved to a safe area.

### 2. Operation of Medical Cart

The operation and action of the medical cart 100 having the above-described configuration will be described.

### Flow of Operation

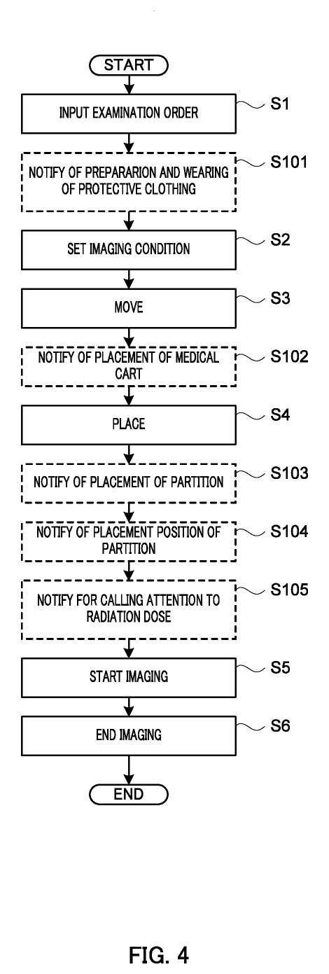

FIG. 4 is a flowchart for explaining an example of a flow of operation of the medical cart 100 according to the present embodiment. Note that the processing indicated by the broken line in FIG. 4 (step S101 to step S105) is processing relating to notification performed as necessary. That is, the processes of steps S101 to S105 are not performed in some cases depending on conditions, and therefore, these processes are skipped in such a case. Here, the processing of step S1 to step S6 indicated by solid lines will be described, and the processing related to the notification indicated in step S101 to step S105 will be described in detail in notification examples 1 to 4 described later.

First, in step S1, an examination order is input from the RIS or the like to the medical cart 100. When the examination order is input, in step S2, the medical cart 100 sets imaging conditions based on the input examination order. Thus, various settings related to the radiographic imaging for the subject by the medical cart 100 are performed.

After the imaging conditions are set and an operator such as a technician confirms various information on radiographic imaging including moving image capturing according to the examination order and the imaging conditions, the operator moves the medical cart 100 to an imaging location in step S3.

When the medical cart 100 is moved, the arm 130 and the input/output section 141 are operated so that the radiation generating apparatus 110 is positioned above the main body section 140, which is the home position. Specifically, as shown in FIG. 2, the rear surface side of the input/output section 141 is in contact with the main body section 140, that is, the inclination angle with respect to the main body section 140 is substantially 0°. In addition, the vertical arm 131 is rotated by 180° with respect to the Y-axis as compared to when the medical cart 100 is used.

By reconfiguring the medical cart 100 in this way, it is possible to appropriately move the medical cart 100 while stabilizing the position of the center of gravity and preventing the radiation generating apparatus 110 from coming into contact with surrounding objects.

Here, the current position of the medical cart 100 may be determined before the medical cart 100 moves to the imaging location. Specifically, for example, if a visible-light camera is mounted on the medical cart 100, the controller 330 captures an image of an area in front of the medical cart 100 in the traveling direction using the visible-light camera. The visible light camera may be a fisheye camera capable of capturing an image of the surroundings of the medical cart 100. The controller 330 identifies a room number or a room name such as ICU or Neonatal Intensive Care Unit (NICU) from the captured visible light camera image by image processing, and acquires the obtained information as current position information.

Furthermore, the controller 330 acquires the floor plan of the room that is the imaging location, using the current location information such as the room number and the room name. The floor plan of the room can be acquired from, for example, a database in which the floor plan of each room is stored. Such a database may be, for example, mounted on the medical cart 100 in advance, or placed in an external system or the like other than the medical cart 100. In a case where the database is placed outside, the controller 330 may acquire information indicating the floor plan of the room serving as the imaging location via the communication section 144.

Note that when the user moves to a plurality of places in order and performs a series of visit examinations, the controller 330 acquires information about the imaging locations based on information included in an examination order corresponding to the series of visit examinations to be performed.

The examination order includes patient information on a patient who is a target of the visit examination and information on a room which is an imaging location such as a room name and a bed number. Therefore, in this case, it is preferable that an examination to be performed most recently can be selected from examination orders corresponding to a series of visit examinations and, when the examination is selected, the controller 330 acquires information on the patient name and the imaging location associated with the selected examination and causes the input/output section 141 to display the information.

Here, the imaging location acquired at this time is the destination of the medical cart 100. Therefore, for example, even when the current position of the medical cart 100 is unknown, desired room layout data can be acquired by acquiring room layout data from the database using the information on the imaging location.

At this time, if the tube side input/output section serving as the input/output section 141 is placed in the radiation generating apparatus 110, the controller 330 may control the tube side input/output section to display the room layout diagram during the movement of the medical cart 100. Thus, the operator or the like can consider the placement of the medical cart 100 while moving.

Further, the room layout diagram includes arrangement information for each bed. The controller 330 recognizes the bed arrangement of the patient in the room using the bed number included in the selected imaging location of the patient, and causes the bed arrangement to be displayed in an overlapping manner on the room layout diagram. Thus, the operator or the like can make a more detailed study while moving.

Furthermore, in this case, information on the presence or absence of a patient other than the imaging target patient in the room that is the imaging location may also be displayed. Specifically, for example, the controller 330 refers to the database on the bed usage of the patient using the information on the imaging location, acquires the information on the usage states of all the beds in the corresponding room, and causes the information to be displayed in an overlapping manner on the room layout diagram. Alternatively, in the controller 330, the beds may be limited to those which are around the imaging target patient. Furthermore, the controller 330 may target a bed at least a part of which is included in a predetermined surrounding area (e.g., within about 2 m to about 3 m) of the bed of the patient to be imaged. Thus, the user can grasp in advance the presence or absence of a patient who is likely to be exposed to radiation in the surroundings of the patient to be imaged, and can consider countermeasures against ambient exposure.

Upon arrival at the imaging location, in step S4, the operator places the medical cart 100 at a predetermined position based on the information on the imaging location. The information on the imaging location is included in the examination order or the imaging conditions.

Next, when the placement of the medical cart 100 is completed, in step S5, the operator operates the medical cart 100 to start radiographic imaging. When the radiographic imaging is completed, the operator checks the imaging data, and determines the necessity of re-imaging based on the imaging data, for example. If re-imaging is necessary, radiographic imaging is performed again, and if re-imaging is unnecessary, radiographic imaging ends in step S6.

### Notification

Since radiographic imaging using the medical cart 100 is performed in an ordinary room such as a general hospital room, there is a possibility that a patient, a nurse, or the like other than the subject such as a technician is also exposed to radiation. Therefore, in the present embodiment, various notifications for, for example, appropriately performing radiographic imaging while suppressing exposure of nearby people to radiation are provided in accordance with the behavior of the operator who operates the medical cart 100 or the like.

In the present embodiment, the medical cart 100 generates and presents a necessary notification based on the information on the operation process of the medical cart 100. The notification to the operator or the like in this case is provided, for example, using the input/output section 141 (the output section 320). The notification may be a simple instruction to the operator, or may cause the operator to confirm whether the notified work has been executed. In addition, the notification may be displayed on the screen of the input/output section 141 (output section 320) or may be output by voice.

Hereinafter, specific notification examples by the medical cart 100 will be described.

### Notification Example 1: Preparation or Wearing of Protective Clothing

A notification example 1 is a notification related to "preparation or wearing of protective clothing". This notification is performed by the process shown in step S101 of FIG. 4. As described above, radiographic imaging using the medical cart 100 is performed in a normal room such as a general hospital room. At this time, the radiographic imaging is performed by an operator, such as a technician, operating the medical cart 100. Therefore, in this case, the operator who operates the medical cart 100 may also be exposed to radiation.

Therefore, in the present exemplary embodiment, in order to suppress exposure of the operator to radiation, a notification prompting the operator to prepare and wear protective clothing is performed.

To be specific, in step S101 of FIG. 4, the controller 330 of the medical cart 100 determines that radiographic imaging is to be performed in response to input of an examination order from the RIS or the like via the communication section 144 (step S1 of FIG. 4). Then, the controller 330 provides, based on the determination result, a notification for prompting the operator to prepare or wear protective clothing.

Note that the visit examination in which the radiographic imaging is performed by the medical cart 100 is not only performed on one patient at a specific place, but also may be performed by sequentially moving to a plurality of places and sequentially moving to the patients at the respective places. In such a case, an order of performing a series of visit examinations is selected from the examination orders, and a group of visit examinations to be collectively performed is created. Then, the visit examination is performed on each patient. In this case, upon selection of an examination order or creation of a visit examination group, the controller 330 may determine that a series of visit examinations, that is, radiographic imaging will be performed.

Here, operation information corresponding to an operation or an input related to determination of an examination order, such as an input of an examination order by an operator or the like or an operation for creating a visit examination group, corresponds to information related to an operation process. Therefore, when determining that an examination order has been input based on the information on the operation process, the controller 330 provides a notification for prompting the user to prepare or wear protective clothing in step S101.

Furthermore, the controller 330 may provide the notification prompting the operator to prepare or wear protective clothing, based on the determination result as to whether or not the series of visit examinations is to be performed. For example, the operation or input related to the determination of the examination order may be performed for the purpose of organizing examination orders, and the actual visit examination may be performed later. In such a case, as in step S101 of FIG. 4, if a notification for prompting preparation or wearing of protective clothing is provided in response to an operation or an input related to determination of an examination order, the notification is a notification provided at an inappropriate timing, and thus convenience is reduced.

Therefore, for example, an operation element such as a button that allows the operator or the like to inform the medical cart 100 of the start of the visit examination may be prepared in advance, and whether or not the series of visit examinations is to be performed may be determined in response to the operator's operation on the operation element. Here, the operation information according to the operation related to the execution of the series of visit examinations such as the operation to the operation element described above or the information indicating the determination result of whether or not the series of visit examinations is executed corresponds to the information related to the operation process. If the controller 330 determines, based on the information about the operation process, that a series of visit examinations will be performed, the controller 330 provides a notification prompting the user to prepare or wear protective clothing in the step S101.

At this time, the controller 330 may determine radiation protection means to be prepared, such as protective clothing, in accordance with the type of radiographic imaging included in the series of visit examinations. For example, still image capturing and dynamic imaging can be considered as types of radiographic imaging. In dynamic imaging, an exposure dose to the surroundings per imaging tends to be larger than that in still image capturing. Therefore, the operator wants to more reliably suppress exposure during dynamic imaging than during still image capturing. Therefore, the controller 330 may notify the operator to prepare or wear protective clothing so that the operator uses the protective clothing having a higher protective performance than that in the still image capturing or uses pieces of protective clothing in an overlapping manner in the dynamic image capturing. As will be described in another notification example below, the controller 330 may issue a notification to use a partition serving as a protective material for blocking radiation during dynamic imaging.

However, when each of the protection means to be used is prepared in accordance with the imaging type in the series of visit examinations, there is a disadvantage that the number and weight of the protection means prepared for the series of visit examinations are increased or workability is lowered.

Therefore, in this case, the protection level is associated with the imaging type, the radiation irradiation condition, and the like in advance, and the protection level is set for the visit examination that is actually performed. Then, the controller 330 determines the protection level for the visit examination based on the imaging type, the irradiation condition, and the like included in the visit examination. At this time, the controller 330 may switch protection means to be used, such as protective clothing and a partition, based on the determination result.

For example, the protection level of a visit examination including dynamic imaging is set higher than that of a visit examination not including dynamic imaging. Therefore, in this case, the controller 330 issues a notification to prepare a partition as the protection means, for example.

Note that the protection level may not be set for the visit examination, and the protection means may be directly set according to the imaging type, the irradiation conditions, and the like included in the visit examination.

In this way, the protection means is switched according to the imaging type or the like included in the visit examination, and thus it is possible to appropriately set the balance between the risk of radiation exposure and the workability.

The types of radiographic imaging included in the series of visit examinations are determined by the setting of the imaging conditions in step S2. Therefore, the processing in step S101 may be performed after the processing in step S2.

In addition, the notification to the operator or the like is performed by using the input/output section 141 (output section 320) as described above. However, at this time, it may be difficult to appropriately recognize which protection means is specifically indicated only by information by characters or voice. Therefore, in a case where the notification is performed with respect to the operator or the like, not only the character or the voice but also an image (an illustration, a photograph, or the like) of the protective clothing to be prepared may be displayed.

The image to be displayed at this time can be registered in advance by a user or an operator, for example. Thus, the actually used protective means such as pieces of protective clothing and partitions can be displayed, so that the prepared protective means can be easily recognized.

In a case of displaying the image of the protection means, for example, the image may be popped up on the display screen so that the image can be erased by pressing an OK button. However, the display screen may display other information, such as confirming order information during preparation for a series of visit examinations. Therefore, for example, it is preferable that the screen can be switched by a tab and an image of the protection means can be displayed by selecting the tab because workability is improved.

In addition, the controller 330 may confirm the presence or absence of a non-target patient who may be exposed to radiation around the patient based on the information indicating the imaging location of the target patient, and reflect it on the display of the protection means to be prepared. For example, when there is no other patient, who is not an imaging target, within 3 m of the imaging target patient, the partition may not be necessary. In this case, it is possible to reduce the burden on the operator or the like by excluding the partition as the protection means from the protection means to be prepared.

Various displays are normally displayed on the input/output section 141 (output section 320) of the medical cart 100. On the other hand, the radiation generating apparatus 110 is located at its home position while the medical cart 100 is moving or the like (see FIG. 2). In this case, since the radiation generating apparatus 110 is placed on the upper side of the input/output section 141, a display displayed on the input/output section 141 is less likely to be visually recognized.

Therefore, in a case where the tube side input/output section that is the input/output section 141 is placed in the radiation generating apparatus 110, it is more preferable to display the image or the like of the protection means on the tube side input/output section. Thus, the operator or the like can prepare for a protection means with the radiation generating apparatus 110 located at the home position.

Note that in a case where the tube side input/output section is not placed, the display of the image or the like of the protection means may be displayed at one or more places of four corner regions (e.g., a region of about 15 cm × 15 cm) of the input/output section 141 (the output section 320) in the input/output section 141. Alternatively, the image or the like of the protection means may be displayed in a predetermined region located inward from the outer periphery of the input/output section 141 (output section 320) by a predetermined distance (e.g., about 15 cm).

This is because, at the time of movement or the like, the visibility of the vicinity of the center of the input/output section 141 is deteriorated as compared with the time of imaging or the like, but the visibility of the vicinity of the outer periphery of the input/output section 141 is relatively ensured. Thus, even when the tube side input/output section is not placed, the operator or the like can prepare for the protection means with the radiation generating apparatus 110 placed at the home position.

Alternatively, the information may be displayed on an external portable terminal such as a tablet or a smartphone that can communicate with the medical cart 100.

The display of the image or the like of the protection means may be automatically terminated and erased at the timing when the preparation of the operator or the like is terminated. The timing at which the preparation of the operator or the like is completed is determined based on, for example, the start of movement of the medical cart 100. This is because the movement of the medical cart 100 means that the preparation of the protection means is completed.

The movement of the medical cart 100 is determined based on the information on the movement of the medical cart 100. The information related to the movement of the medical cart 100 is, for example, driving information or braking information related to the movement of the medical cart 100, acceleration sensor information attached to the medical cart 100, and acceleration sensor information mounted on the radiation detection apparatus 120 connected to the medical cart 100.

In this case, based on the information on the movement of the medical cart 100, for example, when the moving speed of the medical cart 100 reaches a certain level or higher, when the movement of the medical cart 100 is started, when the driving of the movement of the medical cart 100 is started, or when the brake of the medical cart 100 is released, the controller 330 determines that the preparation of the operator or the like is completed. Then, the controller 330 erases the image or the like of the protection means displayed on the input/output section 141.

### Notification Example 2; Proposal of Arrangement Position

The notification example 2 is a notification for suggesting the "placement position of the medical cart 100". This notification is performed by the process shown in step S102 of FIG. 4. The medical cart 100 is preferably placed in the vicinity of a subject on which radiographic imaging is appropriately performed. On the other hand, when radiographic imaging is performed in a normal room, the layout such as the arrangement of beds may vary depending on the imaging location. Therefore, it is difficult to uniquely determine the placement of the medical cart 100.

Therefore, in the present embodiment, when the medical cart 100 arrives at the imaging location, a notification for proposing a placement position of the medical cart 100 is provided in order to appropriately place the medical cart 100 according to the layout of the imaging location.

To be specific, when the movement of the medical cart 100 is started (step S3 in FIG. 4), the controller 330 acquires position information indicating the current position of the medical cart 100. The position information is acquired by using, for example, a Global Navigation Satellite System (GNSS) such as a Global Positioning System (GPS). Note that the position information is not limited thereto and may be acquired based on a visible-light camera image captured by a visible-light camera, for example, when the visible-light camera is mounted on the medical cart 100.

Upon acquiring the position information, the controller 330 determines whether the medical cart 100 has arrived at the imaging location based on the acquired position information and the information about the imaging location. The information on the imaging location includes position information indicating the position of the imaging location and information on the imaging location such as a layout.

More specifically, the controller 330 determines, based on the relationship between the current position of the medical cart 100 indicated by the position information and the position of the imaging location included in the information on the imaging location, whether the medical cart 100 has arrived at the imaging location. Next, upon determination that the medical cart 100 has arrived at the imaging location, in step S102 of FIG. 4, the controller 330 provides a notification for proposing a placement position of the medical cart 100 suitable for imaging. At this time, the controller 330 estimates the placement position of the medical cart 100 based on, for example, information indicating the layout of the imaging location included in the information about the imaging location. Here, the information indicating that the medical cart 100 has arrived at the imaging location corresponds to the information on the operation process.

Note that the information indicating that the medical cart 100 has arrived at the imaging location is not limited to this example. For example, operation information corresponding to a specific operation performed by the operator on the medical cart 100 may be information indicating that the medical cart 100 has arrived at the imaging location and may correspond to the information about the operation process.

For example, an operation element such as a button to be operated when the medical cart 100 arrives at the imaging location is prepared in advance. Then, when the medical cart 100 arrives at the imaging location and the operator or the like operates the operation element, the controller 330 may determine that the medical cart 100 has arrived at the imaging location. In this case, the operation information responsive to an operation on the operation element indicating arrival is information indicating that the medical cart 100 has arrived at the imaging location and corresponds to the information on the operation process.

Further, for example, while moving to the imaging location, the controller 330 causes the input/output section 141 to display an in-hospital map. Next, when a room that is an imaging location in the in-hospital map is selected by the operator, the controller 330 may determine that the medical cart 100 has arrived at the imaging location. In this case, the operation information responsive to the operator's operation of selecting a room is information indicating that the medical cart 100 has arrived at the imaging location and corresponds to the information on the operation process.

Furthermore, for example, the controller 330 causes the input/output section 141 to display information including one or more patients who are imaging targets when a series of visit examinations are performed. Next, when the patient to be imaged is selected by the operator from the information displayed on the input/output section 141, the controller 330 may determine that the medical cart 100 has arrived at the imaging location or will arrive soon. In this case, the operation information responsive to the operator's operation of selecting a patient is information indicating that the medical cart 100 has arrived at the imaging location and corresponds to the information on the operation process.

The information indicating that the medical cart 100 has arrived at the imaging location is not limited to the operation information according to the specific operation performed by the operator on the medical cart 100. For example, when information about the travel of the medical cart 100 changes, such as a decrease in the travel speed of the medical cart 100 or stoppage of the medical cart 100 for a predetermined time or more, the controller 330 may determine that the medical cart 100 has arrived at the imaging location. In this case, the information on the travel of the medical cart 100 is information indicating that the medical cart 100 has arrived at the imaging location, and corresponds to the information on the operation process.

Furthermore, the condition for the controller 330 to determine the arrival at the imaging location may be a combination of the above-described examples.

Here, for example, in a case where a plurality of patients use one room that is an imaging location, the medical cart 100 is preferably disposed at a position in consideration of exposure to a non-imaging target patient who is not an imaging target. This is because it is possible to reduce radiation exposure to a non-imaging target patient. For example, when the imaging target patient and the non-imaging target patient use two adjacent beds arranged side by side, the medical cart 100 is placed in a passage between the two beds. As a result, the medical cart 100 serves as a shield, and it is possible to reduce radiation exposure to the non-imaging target patient.

In a case where an object or the like serving as an obstacle is placed in the passage between the two beds, it might be unable to place the medical cart 100 between the two beds, and the radiation exposure of the non-imaging target patient may not be appropriately reduced. Therefore, depending on the situation of the room, it may be more efficient to perform imaging with the medical cart 100 placed in a place other than the aisle.

The determination of where to place the medical cart 100 also depends on the magnitude of the effect of ambient exposure, which varies depending on the imaging type. For example, in still image capturing in which the influence of ambient exposure is relatively small, even if priority is given to workability, the influence of exposure is slight. On the other hand, in dynamic imaging in which the influence of ambient exposure is relatively large, it is necessary to prioritize a reduction in exposure over workability.

Therefore, in this case, the suggestion of the placement position of the medical cart 100 may be switched based on one or more imaging types included in the selected examination. For example, when the imaging type included in the examination is only still image capturing, the indication of the placement position of the medical cart 100 is not performed. On the other hand, when at least one dynamic imaging is included as the imaging type included in the examination, it is proposed to place the medical cart 100 in the passage between the two beds.

Note that if the bed adjacent to the bed used by the imaging target patient is not in use (with reference to the database for information indicating whether the bed is in use), it is not necessary to take measures against exposure of surrounding patients to radiation. Therefore, even if the imaging type includes dynamic imaging, the placement position of the medical cart 100 may not be indicated.

The proposal of the placement position of the medical cart 100 can be easily understood by displaying a diagram showing the placement of the target medical cart 100 on the base map in an overlapping manner on the input/output section 141. The current placement of the medical cart 100 may also be displayed. Thus, since the target placement position and the current position of the medical cart 100 are displayed at the same time, usability is improved.

Although the input/output section 141 may be used as the display section, the radiation generating apparatus 110 is often located at a home position during movement or placement operation. Therefore, in a case where the tube side input/output section is placed, it is preferable that the tube side input/output section is used.

Note that similarly to the display of the image of the protection means or the like in the notification example 1, the display of the notification suggesting the placement position is preferably performed on the tube side input/output section in a case where the tube side input/output section is placed.

In addition, even in a case where the tube side input/output section is not disposed, similarly to the notification example 1, the display may be performed at any one or more places of four corner regions (e.g., a region of about 15 cm × 15 cm) of the output section 320 in the input/output section 141, or a predetermined region located inward from the outer periphery of the input/output section 141 by a predetermined distance (e.g., about 15 cm).

Further, the information may be displayed on an external portable terminal such as a tablet or a smartphone that can communicate with the medical cart 100.

Note that the determination of the position of the medical cart 100 is not limited to this example. For example, the controller 330 may determine the position of the medical cart 100 based on information acquired from a camera pre-mounted on the medical cart 100. Further, for example, the controller 330 may determine the position of the medical cart 100 when the medical cart 100 receives a beacon placed in the hospital.

Specifically, for example, when a visible light camera is mounted on the medical cart 100, the controller 330 captures an image of the surroundings of the medical cart 100 using the visible light camera. Further, the controller 330 detects surrounding objects such as a bed, a curtain, a cabinet, and a wall from the captured visible light camera image, and estimates the positional relationship between the medical cart 100 and the detected surrounding objects.

Then, the controller 330 compares the positional relationship with surrounding objects with the layout of the room as the imaging location acquired from the database, and estimates the position, orientation, and the like of the medical cart 100 in the room.

Here, generally, in many facilities, a name card on which the name of a patient is written is posted around a bed. Therefore, the controller 330 may detect the name card from the image captured by the visible light camera and use information obtained by recognizing the name of the patient from the detected name card to determine the position of the medical cart 100.

In this case, the controller 330 can determine the position of the medical cart 100 by acquiring, from the database, the position information on the bed of the patient recognized from the name card. In addition, the controller 330 can accurately determine the position of the medical cart 100 by also using data indicating the layout of the room.

### Notification Example 3: Arrangement of Partition (Protective Material)

Notification example 3 is a notification regarding "arrangement of partitions". This notification is performed by the process shown in step S103 of FIG. 4. When radiographic imaging is performed in a normal room, there is a possibility that surrounding persons other than the subject are exposed to radiation. Therefore, in order to suppress exposure to persons other than the subject, a partition for preventing radiation is preferably disposed between the medical cart 100 and surrounding persons. Therefore, in the present embodiment, a notification for arranging a partition is provided in order to suppress exposure to surrounding persons other than the subject.

Specifically, the controller 330 acquires information indicating the state of the medical cart 100. The information indicating the state of the medical cart 100 is, for example, information about traveling of the medical cart 100 or information indicating whether the radiation generating apparatus 110 mounted on the medical cart 100 is placed at the home position.

Then, the controller 330 determines, based on the information indicating the state of the medical cart 100, whether the medical cart 100 has been placed. More specifically, for example, based on whether the radiation generating apparatus 110 mounted on the medical cart 100 is placed at the home position while the medical cart 100 is stopped, the controller 330 determines whether the medical cart 100 is placed. In this case, when the radiation generating apparatus 110 is not placed at the home position, the controller 330 determines that the medical cart 100 has been placed.

When determining that the medical cart 100 has been placed, the controller 330 provides a notification prompting the user to place a partition in step S103 in FIG. 4. At this time, the controller 330 determines, for example, based on the amount of radiation at the time of radiographic imaging included in the imaging conditions, whether a partition is necessary. Furthermore, the controller 330 estimates the placement position of the partition, for example, based on the position information on the medical cart 100 and the radiation amount at the time of radiographic imaging.

Instead of the radiation dose at the time of radiographic imaging, the type of radiographic imaging to be performed may be used. For example, still image capturing and dynamic imaging can be considered as types of radiographic imaging. In the dynamic imaging, since an exposure dose to the surroundings per imaging tends to be larger than that in the still image capturing, arrangement of the partitions is prompted in the dynamic imaging, and arrangement is not prompted in the still image capturing. Alternatively, an imaged site for radiographic imaging may be used. Generally, a trunk portion such as a lumbar vertebra tends to be imaged with a high dose, and a hand, a foot, and the like tend to be imaged with a low dose. Therefore, arrangement of the partition is prompted in an imaged site to be imaged with a high dose, and arrangement of the partition is not prompted in an imaged site to be imaged with a low dose. Note that the type of radiographic imaging and the imaged site may be combined. Further, information on the necessity of the partition may be attached in advance to the information on the visit examination to be performed, and the attached information on the necessity of the partition may be used.

Here, the information indicating that the medical cart 100 has been placed corresponds to the information on the operation process. The information indicating that the radiation generating apparatus 110 is not placed at the home position corresponds to the information indicating that the medical cart 100 has been placed.

Here, simply disposing the partition might not be able to appropriately suppress exposure to surrounding persons. This is because an appropriate position at which the partition is disposed varies depending on the placement position of the medical cart 100 in the imaging location, the layout of the room, the scattered radiation dose when the subject is irradiated with radiation, and the like.

Therefore, in a case of providing notification of the placement of the partition, notification for proposing an appropriate placement position of the partition may be provided in order to more appropriately suppress exposure to persons other than the subject. This notification is performed by the process shown in step S104 of FIG. 4.

In this case, when the controller 330 provides the notification prompting the arrangement of the partition, the scattered radiation dose is estimated in step S104 of FIG. 4 by simulation based on imaging conditions set for the radiation generating apparatus 110, such as imaging time, frame rate, tube voltage, tube current, radiation time (irradiation time) per frame, tube current-time product (mAs), allowable number of frames (maximum number of frames that can be imaged per imaging), pulse width, pulse interval, pulse period, and pulse duty ratio. The controller 330 may estimate the scattered radiation dose in consideration of the layout of the imaging location, the material of the bed, the imaged site (chest, abdomen, limbs, or the like), the irradiation direction of radiation, the body thickness, the position (orientation) of the arm, the positions (layout) of the medical cart and the bed, the positions of another monitor such as a respiration monitor, and the like.

Next, the controller 330 notifies of (presents) an appropriate placement position of the partition based on the estimated scattered radiation dose and the information about the imaging location. Here, the generation of the notification for prompting the arrangement of the partition corresponds to the information on the operation process. In this case, the controller 330 estimates the scattered radiation dose of radiation emitted by the radiation generating apparatus 110, and overlaps the estimation result on the base map of the imaging location stored in the memory 142.

In the scattered radiation dose map which the controller 330 causes the input/output section 141 to display, the estimated scattered radiation dose is overlapped on a base map. The controller 330 may cause the estimated result to be displayed two dimensionally or three dimensionally. In addition, the controller 330 estimates a position of a partition capable of appropriately suppressing exposure to a person other than the subject based on the base map with which the scattered radiation doses are overlapped, and further overlaps the partition therewith.

As described above, the controller 330 may notify the surrounding persons to move away from the predetermined range based on the estimated scattered radiation dose. At this time, the controller 330 may switch the separation distance based on the estimated scattered radiation dose.

Further, the controller 330 may switch and display the separation distance based on the imaging type such as the still image capturing and the dynamic imaging. In dynamic imaging, the scattered radiation dose per imaging tends to be larger than in still image capturing. Therefore, the controller 330 may display the separation distance for farther separation in the dynamic imaging than in the still image capturing. To be specific, for example, the controller 330 displays the separation distance as 2 m in the case of still image capturing, and displays the separation distance as 3 m in the case of dynamic imaging.

Here, when an inexperienced user operates the medical cart 100, it may be difficult for the user to intuitively determine how far the separation distance is. Therefore, when the separation distances are displayed, the controller 330 preferably causes a diagram illustrating the separation distances (2 m or 3 m) to be displayed together with a diagram of the medical cart 100 as viewed from above. In addition, in a case where the separation distance is illustrated and displayed, it is more preferable to overlap the separation distance on the acquired base map because the separation distance is clearly indicated.

Furthermore, the medical cart 100 may provide a notification to change the radiation dose, based on the estimated scattered radiation dose and information indicating the condition of the subject included in the imaging conditions.

Furthermore, similarly to the notification example 2, it is preferable that the arrangement of the partitions is performed in consideration of exposure to the non-imaging target patient. This is because it is possible to reduce radiation exposure to a non-imaging target patient. For example, when the imaging target patient and the non-imaging target patient use two adjacent beds arranged side by side, the partition is placed in a passage between the two beds. Thus, it is possible to reduce radiation exposure to a non-imaging target patient.

In a case where an object or the like serving as an obstacle is placed in the passage between the two beds, it might be unable to place the partition between the two beds, and the radiation exposure of the non-imaging target patient may not be appropriately reduced. Therefore, it may be more efficient to arrange the partition at a place other than the aisle depending on a situation of the room.

The determination of where to place the partition also depends on the magnitude of the effect of ambient exposure, which varies depending on the imaging type. Therefore, it is preferable that the proposal of the placement position of the partition be switched based on one or more imaging types included in the selected examination. For example, in a case where the imaging type included in the examination is only still image capturing, the indication of the placement position of the partition may not be performed. On the other hand, in a case where at least one dynamic imaging is included as the imaging type included in the examination, it is proposed to arrange a partition in a passage between two beds.

When the bed adjacent to the bed used by the patient to be imaged is not used with reference to the information stored in the database, it is not necessary to take measures against radiation exposure to surrounding patients. Therefore, even when the imaging type includes the dynamic imaging, the placement position of the partition may not be instructed.

### Notification Example 4; Radiation Dose

A notification example 4 is a notification related to "radiation dose". This notification is performed by the process shown in step S105 of FIG. 4. In general, a radiation dose tends to be larger in radiographic dynamic imaging than in radiographic imaging of a still image. Therefore, it is conceivable that the radiation dose to the subject during radiographic dynamic imaging exceeds the reference amount.

Therefore, in the present embodiment, a notification for calling attention to the fact that the radiation dose to the subject exceeds the reference amount is performed.

To be specific, when the radiographic imaging by the medical cart 100 is started (step S5 in FIG. 4), the controller 330 determines, in step S105 in FIG. 4, based on the imaging conditions, whether the radiation dose during the radiographic imaging exceeds a reference. At this time, when the type of radiographic imaging determined by the setting of the imaging conditions is "dynamic imaging", the radiation dose may exceed the criterion, and thus the controller 330 provides a notification indicating that attention should be paid to the management of radiation. Here, the information on the imaging type determined by the setting of the imaging condition corresponds to the information on the operation process.

As described above, the medical cart 100 that is a mobile radiographic imaging apparatus according to the present embodiment provides a notification for presenting an action taken by an operator based on various information on the operation process of the medical cart 100, such as an examination order, imaging conditions, the position information on the medical cart 100, and the scattered radiation dose. Thus, in the process of operating the medical cart 100, the operator is notified of a necessary action. Therefore, an operator can appropriately work at the time of radiographic imaging.

While the present embodiment has been described above, the present disclosure is not limited to the above-described embodiment, and various modifications and applications may be made therein without departing from the spirit and scope of the present disclosure. For example, although the medical cart 100 that is a mobile radiographic imaging apparatus has been described in the present embodiment, the present invention is not limited thereto, and various notifications may be similarly provided to a stationary radiographic imaging apparatus.

Although embodiments of the present invention have been described and illustrated in detail, the disclosed embodiments are made for purpose of illustration and example only and not limitation. The scope of the present invention should be interpreted by terms of the appended claims.

What is claimed is:

1. A mobile radiographic imaging apparatus that performs radiographic imaging on a subject, the mobile radiographic imaging apparatus comprising:
a hardware processor that generates a notification for presenting an action taken by an operator during the radiographic imaging based on information regarding an operation process of the mobile radiographic imaging apparatus; and
an output section that outputs the notification.

2. The mobile radiographic imaging apparatus according to claim 1, wherein
when, based on operation information corresponding to an operation or input related to determination of an examination order for the subject, the hardware processor determines that the examination order is input, the hardware processor generates the notification for prompting the operator to prepare or wear protective clothing.

3. The mobile radiographic imaging apparatus according to claim 1, wherein
when, based on information indicating that the mobile radiographic imaging apparatus has arrived at an imaging location, the hardware processor determines that the mobile radiographic imaging apparatus has arrived at the imaging location, the hardware processor generates the notification for proposing a placement position of the mobile radiographic imaging apparatus based on the information on the imaging location.

4. The mobile radiographic imaging apparatus according to claim 1, wherein
when, based on information indicating that the mobile radiographic imaging apparatus has been placed, the hardware processor determines that the mobile radiographic imaging apparatus has been placed, the hardware processor generates the notification for prompting placement of a protective material based on any of a radiation dose during the radiographic imaging, a type of the radiographic imaging, an imaged site, or necessity information on the protective material attached to visit examination information.

5. The mobile radiographic imaging apparatus according to claim 4, wherein
when the notification for promoting placement of the protective material is generated, the hardware processor generates the notification for proposing a placement position of the protective material based on position information of the mobile radiographic imaging apparatus, a scattered radiation dose during the radiographic imaging, and information on the imaging location.

6. The mobile radiographic imaging apparatus according to claim 1, wherein
the hardware processor generates, during dynamic imaging, the notification for calling attention to a fact that a radiation dose exceeds a reference amount, based on an imaging condition.

7. A radiographic imaging method for a mobile radiographic imaging apparatus that performs radiographic imaging on a subject, the radiographic imaging method comprising:
generating a notification for presenting an action taken by an operator during the radiographic imaging based on information regarding an operation process of the mobile radiographic imaging apparatus; and
outputting the notification.

8. A non-transitory computer-readable recording medium storing a program that causes a computer to execute the radiographic imaging method according to claim 7.
